(19) United States
(12) Patent Application Publication
(10) Pub. No.: US 2011/0202088 A1
(43) Pub. Date: Aug. 18, 2011

(54) Embolectomy Device With Optional Vibrator

(75) Inventors: Ronen Eckhouse, Yokneam (IL); Shimon Eckouse, Haifa (IL)

(73) Assignee: RAPID MEDICAL LTD., Yokneam (IL)

(21) Appl. No.: 13/125,324

(22) PCT Filed: Oct. 22, 2009
(86) PCT No.: PCT/IL09/00992

§ 371 Date: Apr. 21, 2011

Related U.S. Application Data
(60) Provisional application No. 61/108,238, filed on Oct. 24, 2008.

(51) Int. Cl. A61F 2/01

(52) U.S. Cl. 606/200

(57) ABSTRACT

The present invention provides an embolectomy device for removal of clots from vasculature, said device comprising: a proximal effecter characterized by a non-expanded configuration and an expanded configuration; a distal effecter characterized by a non-expanded configuration and an expanded configuration; said proximal effecter, in said expanded configuration is adapted for grasping a proximal portion of the target embolus; said proximal effecter, in said expanded configuration is adapted for grasping a distal portion of said target embolus; wherein both said effecters, when expanded, are oppositely positioned and are adapted to operate in concert, for trapping said clot such that said clot is (i) manipulatable along and/or around the main longitudinal axis of said vasculature in a predetermined set of motions; (ii) extracted out of said vasculature.

### FIELD OF THE INVENTION

The present invention relates to a minimally invasive or surgical device deliverable through an intravascular catheter to the site of an embolism. It is designed to remove emboli, even when found in the distal, tortuous neurovasculature. The device includes several sections. The device may have a core element, e.g., a core wire. Placed distally on the core element is the distal embolism collection assembly or collector. The distal embolism collection assembly is expandable, potentially self-expanding and may be fixedly, distally joined to the core element or may be movable with respect to the core element. The distal embolism collection element, while in a lower profile or collapsed condition, is passed through the embolism or between the embolism and the vascular wall and then expanded. The device also includes a proximal embolism collection assembly that is expandable. The two embolism collection assemblies are associated with each other in such a way that the two embolism collection assemblies may be moved towards each other to trap the targeted embolism and to separate it from the vascular wall, for removal from the body. Each of the collection assemblies may comprise a leading element and a following or “clean-up” element. The leading element may have one or more functions with regard to a target embolism. The leading element may be configured to macerate or extrude softer portions of the clot and then to allow those resulting “bits” to pass through for collection by the following element. The leading element may also functionally grab and hold onto the comparatively firmer portion of the embolism. The following element is filter-like and collects the portions of targeted embolism assing through the leading element. This structure, in separating the embolism from the vascular wall, substantially lowers the catheter pulling force required to withdraw or extract the clot from its lodgment. Also described are structures for vibrating components in the device, typically the leading and following elements, operative to assist in separating the targeted embolism from the vascular wall prior to withdrawing or extracting the clot from the vascular site.

### BACKGROUND OF THE INVENTION

The present invention provides an invasive embolectomy device designed to extract emboli found in the human vasculature, particularly in the distal, tortuous neurovasculature, with a low or minimized pulling or extractive force. The device catches or nets the embolism between two opposing, multi-element collection assemblies and, in doing so, separates the embolism from the vascular wall. An optional vibrator may assist the device in separating the target embolism from the vascular wall, thereby lowering further the force needed to extract a target clot from the vasculature.

The use of inflatable balloons to remove emboli has been practiced for many years. The “Fogarty catheter” has been used, typically in the peripheral vasculature, to remove clots from arteries found in legs and in arms. These well known devices have been described in some detail in U.S. Pat. No. 3,435,826, to Fogarty and in U.S. Pat. Nos. 4,403,612 and 3,367,101. These patents describe a balloon catheter in which a balloon material is longitudinally stretched when deflated.

Emboli occasionally form around the valves of the heart and then are dislodged and follow the blood flow into the distal regions of the body. They are particularly dangerous if the emboli passes into the neurovascular where it results in an embolic stroke. As will be discussed below, many such occlusions occur in the middle cerebral artery (MCA), although such is not the only site where emboli come to rest. Obviously, when blood flow is inhibited or cut off completely from a portion of the brain, the brain's oxygen supply is limited causing severe problems.

In procedures for removing emboli using the Fogarty catheter or other similar catheters, it is typical, first, to locate the clot using fluoroscopy. The embolectomy catheter is then inserted and directed to the clot. The distal tip of the balloon catheter is then carefully moved through the center of the clot. Once the balloon has passed through to the distal side of the clot, the balloon is inflated. The balloon catheter is then gradually and gently withdrawn. The balloon, in this way, acts to pull the clot ahead of the balloon. The majority of procedures using a Fogarty catheter repeat these steps until the pertinent vessel is cleared of clot material.

Such vaso-occlusions occur in a wide variety of sites within the body. The lodging of thrombus in various sites is complicated by the presence of atherosclerosis. This disease causes the vessels to become tortuous and narrowed. These anomalies are often considered to be the result of the growth of atherosclerotic plaque. Clots occurring in these diseased vessels are difficult to remove using balloon or Fogarty catheters.

Removal of emboli using balloon catheters is rife with potential problems. One such problem occurs during removal of a clot. The resistance to such removal often causes the balloon portion of the catheter to evert over the tip of the catheter. Should the user need to partially deflate the balloon during such a deflation, the distal tip of the balloon may become distended and angulated. Another difficulty with balloon catheters is the possibility of damage to the intima of arteries. Inflation pressures can create forces significant enough to shear such a vessel lining or to dislodge plaque lodged on such a wall. In the worst case, the balloon may rupture leaving balloon detritus in the bloodstream.

Movement of a balloon in the MCA can displace the clot through more proximal branches into other large vessels such as the internal carotid artery (ICA) and then into other vessels.

There are a variety of different devices intended for use in replacing balloon catheters and in using a device other than a balloon catheter in so removing the emboli.

One such device is shown in U.S. Pat. No. 4,030,503 to Clark III. This patent describes a spiral helix affixed to the distal end of a catheter. In particular, the spiral helix is designed to be rotated and pushed forward through the clot. It is said that the helix screws into the clot, and when it is firmly embedded or is past the clot, the catheter is pulled out of the vessel without rotation. The catheter is said to operate like a corkscrew.

A similar catheter is described in U.S. Pat. No. 4,706,671 to Weinrib. This catheter also has a coil section at its distal end. The coil section is said to be stretched initially into a generally linear insertion position for removing inwardly in a vessel. The coil member is then expanded into the form of a hollow conical scoop to then scoop clot material from the blood vessel. The coil member is stiffened by an internal wire which is then removed. The hollow passageway is then filled with a liquid to stiffen the coils. The coils are said to be of an elastomeric material.

U.S. Pat. No. 4,762,130 to Fogarty et al., describes a helical balloon attached to the distal end of a catheter. The helical or bellowed balloon is maintained in a generally linear condition and passed into a clot. Once the catheter balloon within the clot is inflated, the balloon and adjoining clot are removed together.

Another similar device used more to grip and shear atherosclerotic deposits rather than to remove thrombi is described in U.S. Pat. No. 4,890,611 to Monfort et al. This device incorporates a pair of helical wires placed on the distal end of a wire. The flexible wire is pulled against a flexible catheter, the helical loops cut through and is said to retain sections of plaque for removal.

A thrombus extraction system is shown in U.S. Pat. No. 5,011,488, to Ginsberg. In this device, a deflated (but inflatable) balloon having a proximal conic shape is passed through a targeted thrombus. The balloon is then expanded and retracted so that the proximal end pulls the thrombus into contact with an aspirator. The aspirator then removes the thrombotic material from the vessel.

The Ginsburg patent describes an alternative wire-based configuration of the expandable member. In this variation, a wire coil is attached to an extension wire which may be moved between an extended position and a retracted position. The retracted or expanded configuration is illustrated to have a conical shape. The cone's proximal end is shown to be smaller than the distal end.

U.S. Pat. No. 5,112,347, to Taheri, shows an inflatable balloon-type embolectomy catheter. The balloon has a number of fingers arranged in a leaf spring arrangement inside the balloon. The balloon is hydraulically inflated and forms a cone after inflation. The deflated device is shown in FIGS. 11 through 14 to be passed distally past an embolism before inflation. After inflation, the large end of the balloon collects the embolism as it is pulled past the appropriate site in the vessel.

U.S. Pat. No. 5,192,286, to Phan et al., shows a retrieval catheter for removing materials from various body lumens. The retrieval catheter is shown to have a slack net which may be collapsed for passage into lumen past the material to be collected.

The net is unfolded after such passage and materials such as uretral stones are removed.

U.S. Pat. No. 5,411,509, to Hilal, shows an embolectomy catheter having a distal elastomeric foam tip. The foam tip has an actuator means suitable for forming the foam section both longitudinally and radially in response to activation of the actuation. In practice, the catheter tip is pressed past an embolism, inflated, and retracted proximally along with the clot.

U.S. Pat. No. 5,490,859, to Mische et al., shows an intravascular occlusion material removal device having an expandable material removal element made up of a number of wires passing between the two ends of such element, a catheter shaft, a drive shaft for spinning the element within the blood vessel, and a collection portion placed on the material removal element for collecting any occlusion material removed by the expandable material removal element. The drive shaft may be operated by a motor connected to the drive shaft proximate to the proximal end of the drive shaft.

None of above mentioned patents and patent applications discloses a device which is especially adapted to trap a clot such that said clotis (i) manipulatable along and/or around the main longitudinal axis of a vasculature in a predetermined set of motions; such that the clotis extracted out of the vasculature.

### SUMMARY OF THE INVENTION

The present invention provides a minimally invasive medical device deliverable through an intravascular catheter to the site of a resting embolism. It is designed to remove clots from the vasculature, and is particularly useful in removing clots from distal, tortuous vasculature.

This embolectomy device includes several sections. First, the device has a distal collector assembly that, when collapsed, is pushed through the target embolism/clot or between the embolism and the vascular wall. The distal collector assembly is expanded. A proximal collector assembly is then expanded. The distal and proximal collector assemblies are moved towards each other, trapping and holding the clot. One or more components of the device, perhaps the collector assemblies may be vibrated to assist in separating the clot from vascular wall. The delivery microcatheter, along with the two collector assemblies and the trapped clot, are then removed from the vasculature.

It is one object of the present invention to provide an embolectomy device for removal of clot from vasculature, said device comprising:
a. a proximal effecter characterized by a non-expanded configuration and an expanded configuration;
b. a distal effecter characterized by a non-expanded configuration and an expanded configuration;
said proximal effecter, in said expanded configuration is adapted for grasping a proximal portion of the target embolus; said proximal effecter, in said expanded configuration is adapted for grasping a distal portion of said target embolus;
wherein both said effecters, when expanded, are oppositely positioned and are adapted to operate in concert, for trapping said clot such that said clot is (i) manipulatable along and/or around the main longitudinal axis of said vasculature in a predetermined set of motions; (ii) extracted out of said vasculature.

It is another object of the present invention to provide the embolectomy device as defined above, wherein said distal effecter is operative to cross a target clot in said non-expanded configuration and then to be expanded to said expanded configuration. It is another object of the present invention to provide the embolectomy device as defined above, wherein said distal effecter is operative to self-expand from its non-expanded configuration to its expanded configuration.

It is another object of the present invention to provide the embolectomy device as defined above, further comprising a covering net, adapted to accommodate said grasped clot and to extract the same outside the body of the patient.

It is another object of the present invention to provide the embolectomy device as defined above, wherein said net is selected from the group consisting of woven fabrics, non-woven fabrics, gauze-like fabrics, materials having multiple openings, and fluid-permeable membranes.

It is another object of the present invention to provide the embolectomy device as defined above, wherein the distal portion said net is adapted to switch between an expended-configuration to a restricted-configuration; said expended-configuration is operative to allow insertion of at least a portion of a target clot into said net's distal portion; and said restricted configuration is operative to retain said clot within said net. It is another object of the present invention to provide the embolectomy device as defined above, wherein said distal portion of said net is operative to self-expand from its expended-configuration to its restricted-configuration.

It is another object of the present invention to provide the embolectomy device as defined above, wherein said net is an elongated sleeve comprising a restricting sphincter located at the distal end of said sleeve, adapted to retain at least a portion of the target clot within said net.

It is another object of the present invention to provide the embolectomy device as defined above, wherein said distal effecter is incorporated or otherwise connected with said proximal effecter to form a single effecter.

It is another object of the present invention to provide the embolectomy device as defined above, wherein said set of motion is selected from a group consisting of a (i) single reciprocal stroke along the long longitudinal axis of said blood vessel; (ii) a set of reciprocal motions; (iii) a single rotating stroke around said long longitudinal axis of said blood vessel; (iv) a set of rotational motions; (v) vibrating motions; or any combination of the same.

It is another object of the present invention to provide the embolectomy device as defined above, additionally comprising:
an endless wire coupled to a spring-like helical member having a plurality of loops; said loops are positioned at an angle A relatively to the main longitudinal axis of said blood vessel; and, are adapted to radially encircle at least a portion of the outer circumference of said clot; and,
a tubular mesh-like net for both enveloping said helical member and enclosing said clot therein;
wherein at least a portion of said clot is contemporaneously confined within said loops and said mesh-like net, such that radial compression forces and longitudinal shearing forces are exerted on said clot and the tendency of said clot to fragment is mitigated.

It is another object of the present invention to provide the embolectomy device as defined above, wherein said spring-like helical member and/or said mesh-like net comprising means for applying vibration on said clot.

It is another object of the present invention to provide the embolectomy device as defined above, wherein said mesh-like net is made of material selected from a group consisting of woven fabrics, non-woven fabrics, gauze-like fabrics, materials having multiple openings, and fluid-permeable membranes or any combination thereof.

It is another object of the present invention to provide the embolectomy device as defined above, wherein said mesh-like net additionally comprising materials selected from a group consisting of woven fabrics, non-woven fabrics, gauze-like fabrics, materials having multiple openings, and fluid-permeable membranes or any combination thereof.

It is another object of the present invention to provide the embolectomy device as defined above, wherein said spring-like helical member is coupled to said mesh-like net.

It is another object of the present invention to provide the embolectomy device as defined above, to switch from an expended-configuration to a restricted-configuration; said expended-configuration is operative to allow insertion of at least a portion of a target clot in to said mesh-like net's distal portion; and said restricted configuration is operative to retain said clot within said mesh-like net.

It is another object of the present invention to provide the embolectomy device as defined above, wherein said distal portion of said mesh-like net is operative to self-expand from its expended-configuration to its restricted-configuration.

It is another object of the present invention to provide the embolectomy device as defined above, wherein said mesh-like net is an elongated sleeve comprising a restricting sphincter located at the distal end of said sleeve, adapted to retain at least a portion of the target clot within said mesh-like net.

It is another object of the present invention to provide the embolectomy device as defined above, wherein said helical member is characterized by a cross-sectional area selected from a group consisting of circular, oval, round, square, rectangular, triangular, regular or irregular shapes, or any combination thereof.

It is another object of the present invention to provide the embolectomy device as defined above, additionally comprising a motor adapted to rotate said helical member.

It is another object of the present invention to provide the embolectomy device as defined above, wherein said helical member additionally comprising protrusions for assisting in the separation of said clot from said vasculature.

It is another object of the present invention to provide the embolectomy device as defined above, additionally comprising vibration means applied on said clot.

It is another object of the present invention to provide the embolectomy device as defined above, additionally comprising a corkscrew-like effecter, having a coil-like distal end, adapted to penetrate and cross said clot.

It is another object of the present invention to provide the embolectomy device as defined above, wherein the diameter of said helical member is variable so as to better adjust to the diameter of said clot or said blood vessel.

It is another object of the present invention to provide the embolectomy device as defined above, wherein said angle A ranges from about 0 degrees to about 180 degrees.

It is another object of the present invention to provide the embolectomy device as defined above, additionally comprising aspiration means.

It is another object of the present invention to provide the embolectomy device as defined above, wherein at least one of said loops and/or said mesh-like net is coated with lubricious polymeric material selected from hydrophilic polymer material.

It is another object of the present invention to provide the embolectomy device as defined above, wherein said spring-like helical member is characterized by single, double, or triple pitch.

It is another object of the present invention to provide the embolectomy device as defined above, wherein the cross section of at least one of said loops and/or said mesh-like net varies.

It is another object of the present invention to provide a method for removing a clot from blood vessel. The comprising inter alia steps selected from:
i. obtaining an embolectomy device comprising:
   a. a proximal effecter characterized by a non-expanded configuration and an expanded configuration;
   b. a distal effecter characterized by a non-expanded configuration and an expanded configuration;
ii. approximating said clot;
iii. crossing said clot via said distal effecter in said an expanded configuration;
iv. reconfiguring said distal effecter from said an expanded configuration to said expanded configuration;
v. reconfiguring said proximal effecter from said an expanded configuration to said expanded configuration;
vi. grasping both the proximal portion of said clot via said proximal effecter and the distal portion of said clot via said distal effecter;
vii. manipulating said clot along and/or around the main longitudinal axis of said blood vessel in a predetermined set of motions; thereby separating said clot from the walls of said blood vessel; and,
viii. extracting said clot confined from said blood vessel.

It is another object of the present invention to provide the method as defined above, additionally comprising step of selecting said set of motion from a group consisting of (i) a single reciprocal stroke along the long longitudinal axis of said blood vessel; (ii) a set of reciprocal motions; (iii) a single rotating stroke around said long longitudinal axis of said blood vessel; (iv) a set of rotational motions; (v) vibrating motions; or any combination of the same.

It is another object of the present invention to provide the method as defined above, additionally comprising steps of crossing said clot via said distal effecter in said en-expanded configuration; and, expanding to said expanded configuration.

It is another object of the present invention to provide the method as defined above, additionally comprising step of configuring said distal effecter to self-expand from its un expanded configuration to its expanded configuration.

It is another object of the present invention to provide the method as defined above, additionally comprising step of incorporating said distal effecter with said proximal effecter to form a single effecter.

It is another object of the present invention to provide the method as defined above, additionally comprising step of selected the cross sectional area of said helical member from a group consisting of circular, oval, round, square, rectangular, triangular, regular or irregular shapes, or any combination thereof.

It is another object of the present invention to provide the method as defined above, additionally comprising step of penetrating and crossing said clot via a corkscrew-like effecter.

It is another object of the present invention to provide the method as defined above, additionally comprising step of accommodating said grasped clot via a covering mesh-like net.

It is another object of the present invention to provide the method as defined above, additionally comprising step of selecting the material of said mesh-like net from a group consisting of woven fabrics, non-woven fabrics, gauze-like fabrics, materials having multiple openings, and fluid-permeable membranes or any combination thereof.

It is another object of the present invention to provide the method as defined above, additionally comprising step of configuring the distal portion of said mesh-like net to switch from an expended-configuration to a restricted-configuration; said expended-configuration is operative to allow insertion of at least a portion of a target clot into said mesh-like net's distal portion; and said restricted configuration is operative to retain said clot within said mesh-like net.

It is another object of the present invention to provide the method as defined above, additionally comprising step of configuring said mesh-like net to self-expand from said expended-configuration to said restricted-configuration.

It is another object of the present invention to provide the method as defined above, additionally comprising step of retaining at least a portion of the target clot within said mesh-like net.

It is another object of the present invention to provide the method as defined above, additionally comprising step of applying vibration on said clot.

It is another object of the present invention to provide the method as defined above, additionally comprising step of adjusting the diameter of either one of said effecters to the diameter of said clot.

It is another object of the present invention to provide the method as defined above, additionally comprising step of coating at least one selected from a group consisting of said mesh-like net and/or said proximal effecter and/or said distal effecter with lubricious polymeric material selected from hydrophilic polymer material.

It is another object of the present invention to provide an embolectomy device for removal of clots from vasculature, comprising:
an expandable, distal clot collector operative to be pushed through a target clot in a non-expanded condition and then to be expanded to an expanded condition, the distal clot collector operative to capture and to retain at least a portion of the target embolus, and
an expandable, proximal clot collector operative to be delivered to a target clot in a non-expanded condition and then to be expanded to an expanded condition, the proximal clot collector operative to cooperate with the distal clot collector and cooperatively to capture and to retain said at least a portion of the target embolus.

It is another object of the present invention to provide the embolectomy device as defined above, wherein the distal clot collector assembly comprises a leading collector element and a following collector element.

It is another object of the present invention to provide the embolectomy device as defined above, wherein the proximal clot collector assembly comprises a leading collector element and a following collector element.

It is another object of the present invention to provide the embolectomy device as defined above, wherein at least one of the leading collector elements includes openings operative to allow at least a portion of a target thrombus to pass therethrough and where at least one of the following collector elements is operative to retain the at least a portion of the thrombus passing through the at least one of leading collector element.

It is another object of the present invention to provide the embolectomy device as defined above, wherein at least one of the distal clot collector and the proximal clot collector is self-expanding.

It is another object of the present invention to provide the embolectomy device as defined above, wherein at least one of the distal clot collector and the proximal clot collector is not self-expanding.

It is another object of the present invention to provide the embolectomy device as defined above, wherein at least one of the distal clot collector and the proximal clot collector comprises a framework comprising a proximal collapsible ring and at least one rib extending to a central core wire.

It is another object of the present invention to provide the embolectomy device as defined above, wherein at least one of the distal clot collector and the proximal clot collector further comprises a covering selected from the group consisting of woven fabrics, non-woven fabrics, gauze-like fabrics, materials having multiple openings, and fluid-permeable membranes.

It is another object of the present invention to provide the embolectomy device as defined above, wherein the distal clot collector and the proximal clot collector are operative to move towards each other after the distal clot collector and the proximal clot collector are expanded.

It is another object of the present invention to provide the embolectomy device as defined above, wherein the distal clot collector is operative to move towards the proximal clot collector after the distal clot collector and the proximal clot collector are expanded.

It is another object of the present invention to provide the embolectomy device as defined above, wherein the proximal clot collector is operative to move towards the distal clot collector after the distal clot collector and the proximal clot collector are expanded.

It is another object of the present invention to provide the embolectomy device as defined above, further comprising a central core wire affixed to the distal clot collector and extending proximally and operative to move the distal clot collector when expanded.

It is another object of the present invention to provide the embolectomy device as defined above, further comprising at least one vibration component operative to improve removal of a target clot during operation of the device.

It is another object of the present invention to provide the embolectomy device as defined above, further comprising at least one vibration component operative to oscillate at least one of the distal clot collector and the proximal clot collector.

### BRIEF DESCRIPTION OF THE DRAWINGS

FIG. 1 shows a generalized schematic of the arterial system of a human head specifying in particular the position of an embolism in the middle cerebral artery.

FIG. 2 is a view of the system used with the embolectomy device as provided by the present invention to remove an embolus.

FIG. 3 is a partial perspective view of the distal end of one variation of the embolectomy device.

FIG. 4 is a partial cross-sectional view of the distal end of the device in FIG. 3.

FIG. 5A is a partial cross-sectional view of one variation of the device, after expansion.

FIG. 5B is a partial cross-sectional view of a portion of the device shown in FIG. 5A.

FIG. 5C is a partial cross-sectional view of the variation of the device depicted in FIG. 5A, before expansion.

FIG. 6 is a partial cross-sectional view of another variation of the device, after expansion.

FIG. 7A is a partial cross-sectional view of one variation of the device, before expansion.

FIG. 7B is a partial cross-sectional view of the variation of the device depicted in FIG. 7A, after expansion.

FIG. 7C is a perspective partial cross-sectional view of the expandable stop for the device shown in FIGS. 7A and 7B.

FIGS. 8A and 8B show, respectively, front and side views of one variation of collector assemblies.

FIGS. 9A and 9B show, respectively, front and side views of another variation of collector assemblies.

FIG. 10 is a schematic depiction of the passage of soft portions of embolism through a leading element of the embolectomy device.

FIG. 11 is a partial schematic view of certain components of the embolectomy device.

FIG. 12 provides a first exempletive procedure for using the embolectomy device to remove an embolus.

FIG. 13 provides a second exempletive procedure for using the embolectomy device to remove an embolus.

FIG. 14 provides a schematic representation of the device employing optional vibrators.

FIG. 15a to FIG. 15i illustrating in an out-of-scale manner a schematic view (cross section) of a minimally invasive embolectomy device according to another embodiment of the invention.

FIG. 16a to FIG. 16 illustrating a corkscrew-like effecter, having a coil-like distal end, adapted to effectively anchor the clot from its inner portion.

FIG. 17a to FIG. 17b illustrating an externally embolus-warping coil-like effecter.

### DETAIL DESCRIPTION OF THE PREFERRED EMBODIMENTS

The following description is provided, alongside all chapters of the present invention, to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a minimally invasive implement that is designed to restore flow in human vasculature by extraction of clots from the same, even in distal, narrow neurovasculature.

The term “crossing” refers hereinafter to act of intersecting clot and penetrating its interior.

The term “about” refers hereinafter to a range of 25% below or above the referred value.

The present invention provides a minimally invasive implement that is designed to extract clots situated in human vasculature, even in distal, narrow neurovasculature. It is intended to be used rapidly as a flow restoration device. Even in those instances where the embolism/clots is not or cannot be completely removed, the device is believed to be useful in removing a portion of the clot and thereby permitting restoration of partial blood flow. It is suitably flexible to be placed in distal tortuous neurovasculature and hence is useful in treating blocking emboli/clots found there. The device may be considered a partial or complete treatment for embolic stroke.

One object of the present invention is to provide an embolectomy device for removal of emboli/clot from vasculature, said device comprising:
a. a proximal effecter characterized by a non-expanded configuration and an expanded configuration;
b. a distal effecter characterized by a non-expanded configuration and an expanded configuration;
   said proximal effecter, in said expanded configuration is adapted for grasping a proximal portion of the target embolus; said proximal effecter, in said expanded configuration is adapted for grasping a distal portion of said target embolus; wherein both said effecters, when expanded, are oppositely positioned and are adapted to operate in concert, for trapping said clot such that said clot is (i) manipulatable along and/or around the main longitudinal axis of said vasculature in a predetermined set of motions; (ii) extracted out of said vasculature.

Another object of the present invention is to provide a method for removing a clot from blood vessel. The comprising inter alia steps selected from:
i. obtaining an embolectomy device comprising:
   a. a proximal effecter characterized by a non-expanded configuration and an expanded configuration;
   b. a distal effecter characterized by a non-expanded configuration and an expanded configuration;
ii. approximating said clot;
iii. crossing said clot via said distal effecter in said an expanded configuration;
iv. reconfiguring said distal effecter from said an expanded configuration to said expanded configuration;
v. reconfiguring said proximal effecter from said an expanded configuration to said expanded configuration;
vi. grasping both the proximal portion of said clot via said proximal effecter and the distal portion of said clot via said distal effecter;
vii. manipulating said clot along and/or around the main longitudinal axis of said blood vessel in a predetermined set of motions; thereby separating said clot from the walls of said blood vessel; and,
viii. extracting said clot confined from said blood vessel.

In general, the device is used via the steps of penetrating the occlusion (or passing the distal end of the device between the occlusion and the vascular wall), expanding a collector assembly distally of the embolus, expanding a proximal collector assembly, moving the proximal and distal collection assemblies towards each other (optionally with vibration) to separate the embolism/clots from the vascular wall and to grasp the embolus, and removing at least a part of the clot along with the device and its attendant catheter.

The device of the present invention comprises an opposing collector assemblies that are delivered, un-expanded, to the embolism/clots and then situated distally and proximally of the embolism/clot. The collector assemblies are movable with respect to each other to grasp the targeted embolism/clot and to separate the embolism/clot from the vascular wall. One or both of the collector assemblies may be vibrated to assist in removing the clot from the vascular wall. That separation substantially lessen the force needed to withdraw the catheter and to extract the embolism/clots from that vessel. Lowering the force required to move the embolism/clot means that a pull on the catheter is less likely to cause damage to the vasculature.

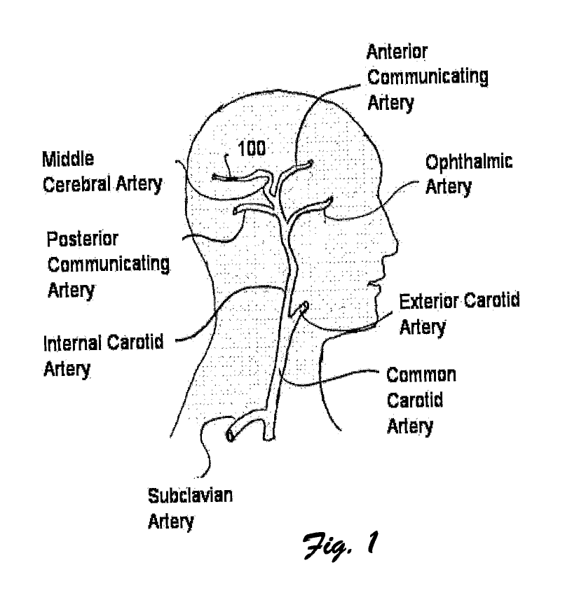

As a matter of practical experience, a large proportion of emboli/clots sloughed into the brain are seldom longer than about 1 to 2 centimeters and, for a variety of reasons both physiological and hemodynamic, often settle in the middle cerebral artery (MCA). As is shown in FIG. 1, the path from the site of origin of a traveling embolus—often the heart—to the MCA is via the common carotid artery past the branch point forming the external and internal carotid arteries into the internal carotid artery (ICA). The MCA is generally considered to be the continuation and termination of the ICA after the siphon and after the branching sizes of a variety of other arteries, e.g., the ophthalmic artery, the anterior communicating artery, the posterior communicating artery, and others. The etiology of such an occlusion is varied, varying, and complicated. The occlusion (100) is shown in the MCA in FIG. 1 at the noted site.

Treatments for such embolic occlusions include catheterization of the patient and introduction of tissue plasminogen activator (TPA), urokinase, or streptokinase to the site of the occlusion. Additionally the embolic occlusion may be penetrated—often with a microcatheter—and the TPA or urokinase or streptokinase introduced distally of the occlusion. Removal of the catheter provides a modest passageway for resumed or increased blood flow past the then-partial occlusion.

The device of the present invention is intended to be used in recanalizing (at least partially) occluded vascular lumen. In general, the device is used via the steps of penetrating the occlusion (or passing the distal end of the device between the occlusion and the vascular wall), expanding a collector assembly distally of the embolism/clots, expanding a proximal collector assembly, moving the proximal and distal collection assemblies towards each other (optionally with vibration) to separate the embolism/clots from the vascular wall and to grasp the embolism/clot, and removing at least a part of the embolism/clot along with the device and its attendant catheter.

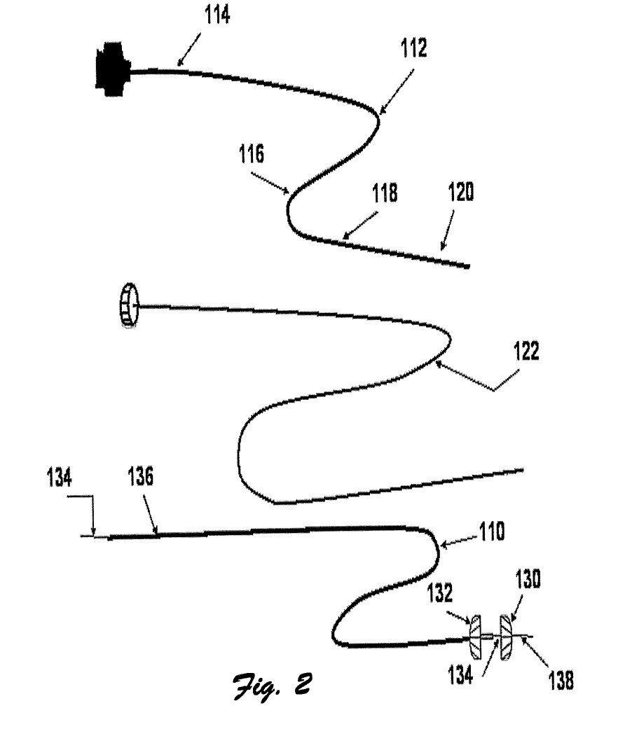

reference is now made to FIG. 2 which is a schematic drawing of the embolectomy device and the major components of the system used to deliver it.

The embolectomy device (110) may be delivered to the target site using an intravascular catheter (112), such as a microcatheter as may be used in neurovascular service. Such a catheter may be made up of a number of sections (114, 116, 118, 120) of different flexibilities. The more-distal sections are typically more flexible and often have decreasing diameters. The most distal section (120) of a suitable microcatheter may be, for instance, 2.0-4.5F, 2.5-3.5F.

A suitable guidewire (122) may be used to deliver the distal end of the catheter proximate to the clot site. The guidewire (122) is then removed from the catheter (112) and the embolectomy device (110) introduced.

The embolectomy device (110) is shown with the distal (130) and proximal (132) collection assemblies in their (post-delivery) expanded condition. The optional nose element (138), used in some variations for penetrating the embolus, is shown distal of the distal collection assembly (130). In this variation, the distal collection assembly (130) is fixedly attached to a core element (134). The core element (134) may be a wire component used to controllably move the distal embolectomy collection assembly (130) with respect to the target clot and to the proximal collection assembly (132).

The proximal collection assembly (132) in this variation is attached to a sleeve (136) that extends proximally; the sleeve (136) is used to deploy the proximal assembly (132) by pushing it from beneath the distal end of the catheter (112). The depicted proximal collection assembly (132) is self expanding. The sleeve (136) and the core element (134) allow differential movement between the distal (130) and proximal (132) collection assemblies in collecting a target embolus.

Other variations of the device include distal and proximal collection assemblies that are configured to move towards each other after expansion without additional intervention by the user, e.g., by a spring or elastic member biased to move distal and proximal collection assemblies towards each other.

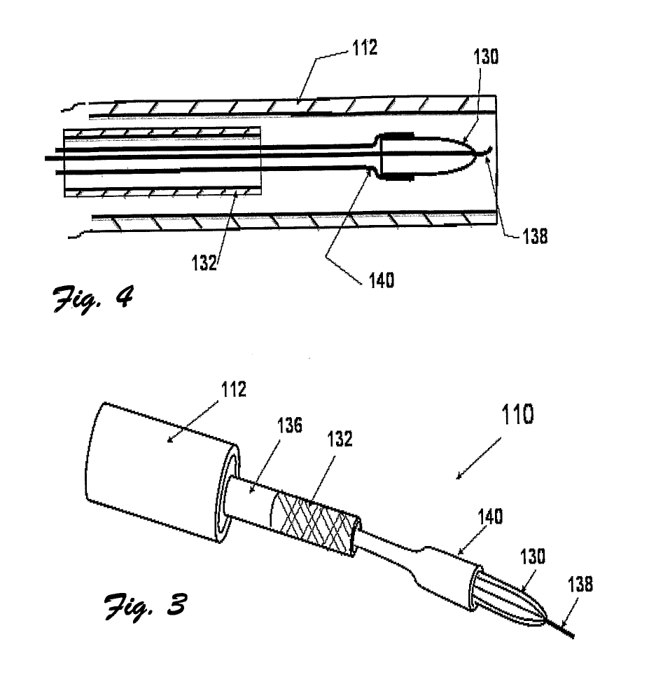

Reference is now made to FIG. 3 which illustrates a perspective view of a variation of the embolectomy device (110) similar to that shown in FIG. 2. FIG. 4 shows a partial, cutaway, side-view of that variation.

In FIG. 3, distal (130) and proximal (132) collection assemblies are shown to have their collapsed, low profile. The proximal collection assembly (132) is shown to be extended from the distal tip of catheter (112), but as mentioned elsewhere, in this variation, the catheter maintains the self-expanding proximal collection assembly (132) in compression before deployment. This compression may be seen with more clarity in FIG. 4. The sleeve (136) connected to proximal collection assembly (132) extends proximally to the user.

The distal collection assembly (130) in this variation may be self-expanding and actuated by withdrawal of the inner deployment sleeve (140).

Although both the distal collection assembly (130) and proximal collection assembly (132) comprise leading and trailing elements (as described elsewhere), the net-like trailing elements are not visible in FIGS. 3 and 4 but are discussed in greater detail below.

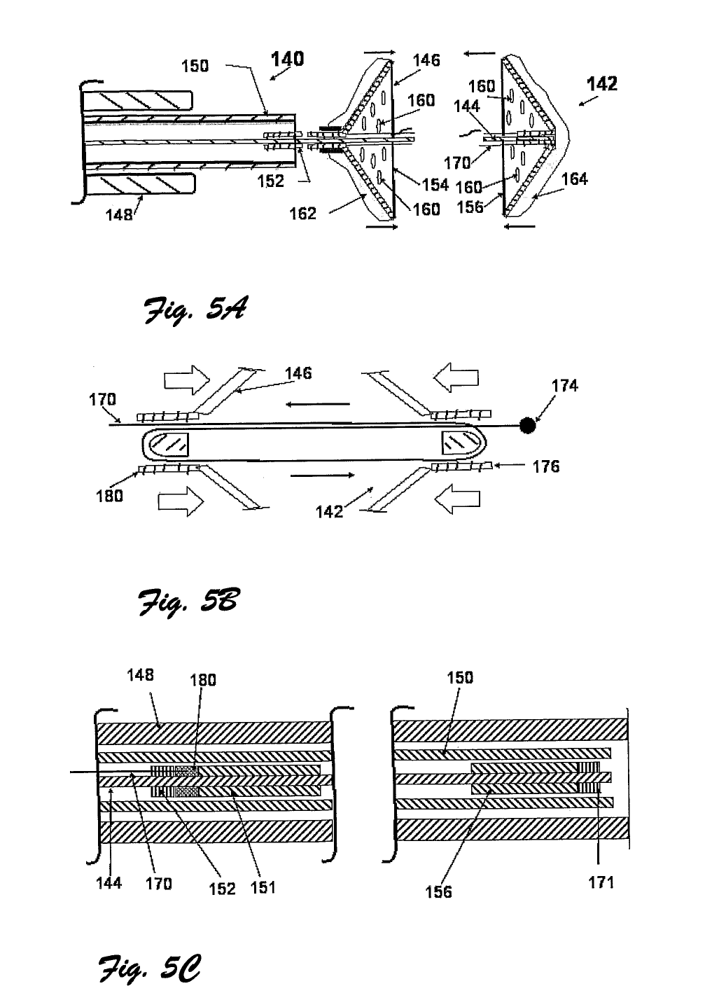

Reference is now made to FIG. 5A which illustrates another variation (140) of the embolism/clots retrieval device. It includes a distal collection assembly (142) (will be referred hereinafter also as distal effecter) fixedly attached to a core wire (144). It further includes a proximal collection assembly (146) (will be referred hereinafter also as proximal effecter) slideable over the core wire (144). The distal end of a delivery catheter (148) may also be seen in the drawing.

This variation of the embolism/clots retrieval device (140) is delivered to the embolism/clot site in the form shown in FIG. 5C, and is discussed below. The delivery release sheath (150) is shown withdrawn into the delivery catheter (148). Withdrawal of the delivery release sheath (150) allows the distal collection assembly (142) and the proximal collection assembly (146) to expand to the active shape seen in the drawings. A proximal limit stop (152) fixedly attached to the core wire (144) limits the proximal movement of that proximal collection assembly (146) on that core wire (144).

The distal collection assembly (142) and the proximal collection assembly (146) each comprise a leading element (154, 156). Due to various physical parameters (e.g., hemodynamics, stagnancy, temperature, etc.), an embolism/clot may vary in consistency. The core volume of a clot is firmest. In the quiescent bloodstream adjacent a clot, blood continues to be added to the clot volume. The later added portion is softest. As shown elsewhere, the leading elements (154, 156) include openings (160) allowing some portion of the softer volume of the embolism/clot to pass therethrough.

The distal collection assembly (142) and the proximal collection assembly (146) also each comprise a following element (162, 164). These following elements (162, 164) are filter-like and entrap any embolic extrudates passing through the leading elements (154, 156).

This variation also includes a cinching line (170) that, when pulled, causes the distal collection assembly (142) and the proximal collection assembly (146) to move towards each other entrapping the embolism/clot between them.

Reference is now made to FIG. 5B which illustrates a schematic illustration of the operation of the cinching line (170). The cinch line (170) is affixed (174) to a center element (176) of the distal collection assembly (142). The cinch line (170) passes from the proximal end through a center element (180) of the proximal collection assembly (146), through the center element (176) of the distal collection assembly (142), again through center element (180) of the proximal collection assembly (146), before being finally affixed (174) to the center element (176) of the distal collection assembly (142).

The cinch line (170) may comprise a high strength, flexible, multifilament or monofilament polymeric cord or thread of a polyamide, e.g., Kevlar, or other suitable material.

As the cinch line (170) is pulled proximally, the two collection assemblies are pulled toward each other separating the clot from the vascular wall and simultaneously entrapping that embolus.

Reference is now made to FIG. 5C which illustrates s a cutaway, side view of the device (140) shown in FIG. 5A, but in an undeployed condition. The delivery release sheath (150) is shown within the delivery catheter (148) covering both the distal collection assembly (142) and the proximal collection assembly (146) and holding them in position prior to expansion. The proximal limit stop (152) fixedly attached to the core wire (144) may also be seen.

Reference is now made to FIG. 6 which illustrate s a schematic representation of another version of a structure and mechanism for controllably pulling the distal and proximal collection assemblies towards each other. As is the case with the device shown in FIGS. 5A-5C, a cinch line (200) passes from the proximal end of the device through the proximal collection assembly (202), through the distal collection assembly (204) (also referred as the distal effecter), and again through the proximal collection assembly (202) (also referred as the proximal effecter),. In this variation, the cinch line (200) is terminated on a floating, radio-opaque clip (206). The clip (206) will stay centered between the distal collection assembly (204) and the proximal collection assembly (202) as the cinch line (200) is pulled. The user may center the radio-opaque clip (206) in the clot and, as the cinch line (200) is pulled, use the clip as a marker to maintain that centered position. The distal collection assembly (204) and the proximal collection assembly (202) approach the clot in equal increments from each side to more effectively separate the clot from the vascular wall without pulling the clot along that wall one way or the other.

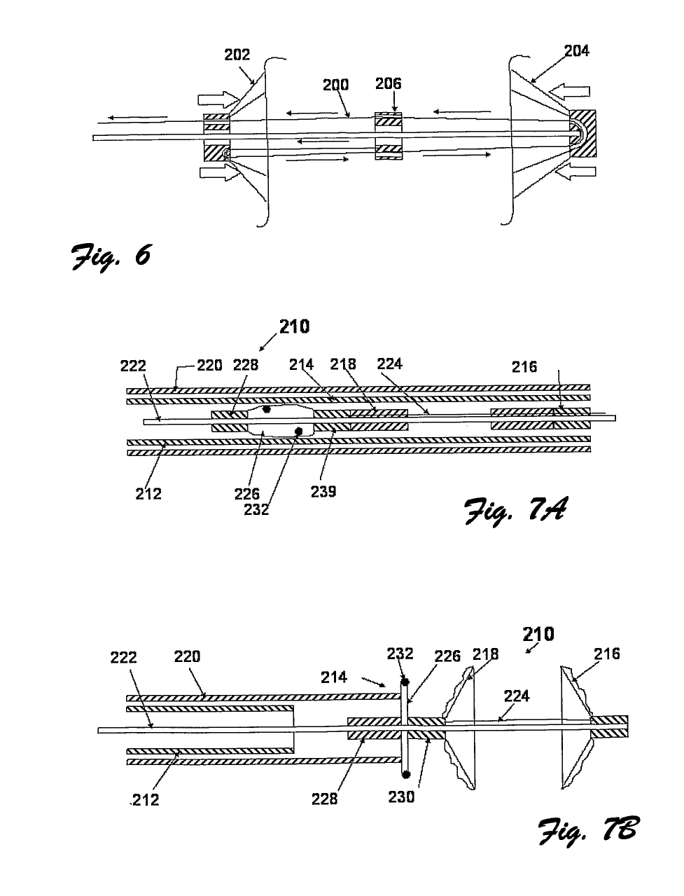

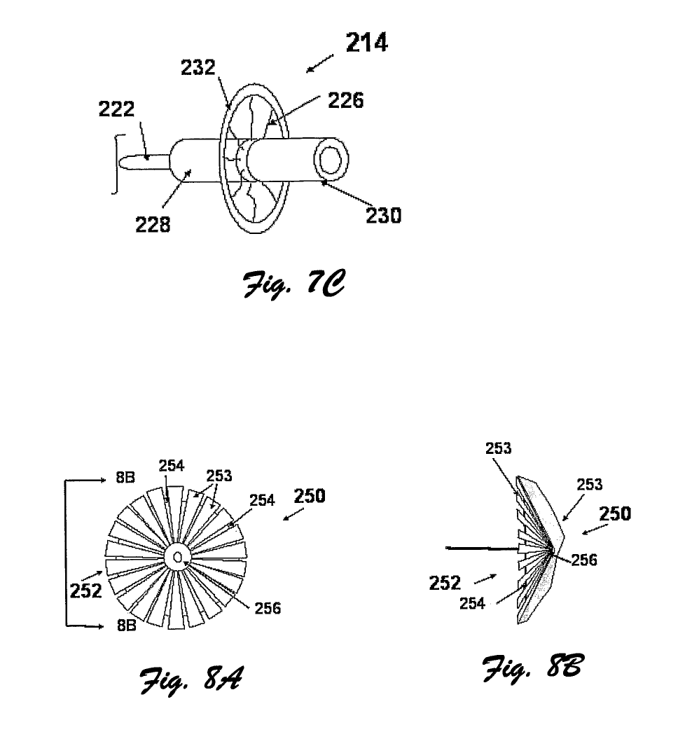

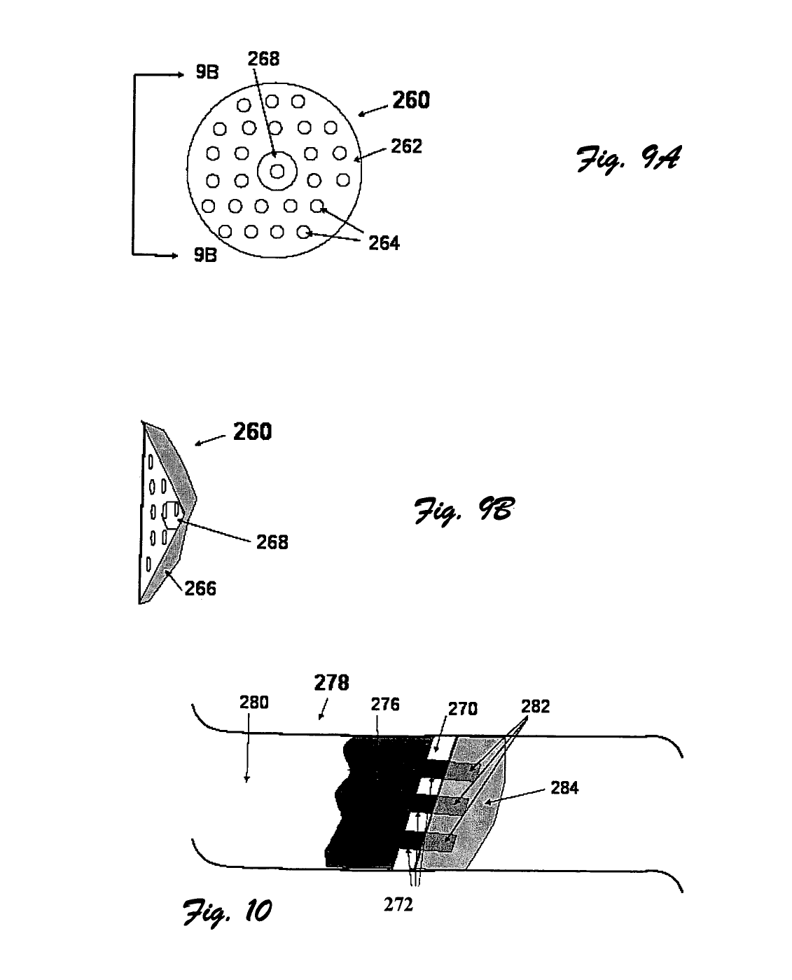

Reference is now made to FIGS. 7A and 7B which illustrates a side view cross sections of another variation of the device (210) in, respectively, the un-expanded condition and as-expanded. This version (210) utilizes a retractable sheath (212) that, after placement, is retracted and allows the distal and proximal collection assemblies (216, 218) to expand. The device uses an expandable “stop” (212) permitting the delivery catheter (220) to be used to control the position of the proximal collection assembly (218) during the step of separating the embolism/clot from the vessel wall and securing it between the distal and proximal collection assemblies (216, 218) (also referred as the proximal and distal effecters respecfully),.

FIG. 7A, in particular, shows the delivery catheter (220) distal tip. In this variation, the delivery catheter (220) is used with a guidewire to penetrate the target embolus. The guidewire is then withdrawn to be replaced by the embolectomy device (210) as shown in the drawing. The delivery catheter (220) is then withdrawn. The embolectomy device (210) is finely positioned and then expanded by withdrawal of the retractable sheath (212).

In FIG. 7A, the distal collection assembly (216) and the proximal collection assembly (218) are shown collapsed onto the core wire (222). The distal collection assembly (216) is affixed to the core wire (222) but the proximal collection assembly (218) is allowed to slide on the core wire (222). In effect, the embolectomy device (210) beneath the retractable sheath (212) is “pulled” into the distal end of the delivery catheter (220) by the core wire (222). The distal collection assembly (216) and the proximal collection assembly (218) may comprise a structure including the leading and following elements discussed elsewhere here.

The proximal collection assembly (218) is held in initial position relative to distal collection assembly (216) by a tether (224). Obviously, the length of the tether (224) specifies the length of the clot to be removed. A long tether (224) means that the proximal collection assembly (218) is a “long” distance from the distal collection assembly (216) and may be used to remove a “long” embolus. When the embolectomy device (210) is pulled into the delivery catheter (220) distal region, the tether (224) pulls the proximal collection assembly (218).

The expandable stop assembly (214) is shown to be affixed to the proximal collection assembly (218) and, in turn, slideable upon the core wire (222). In general, the stop (214) comprises a disc (226) (perhaps of a fabric such as PET, polyethylene or polypropylene (woven or nonwoven), or other foldable, non-tearing material) affixed axially, proximally to a slideable ring (228) and axially, distally to the sliding middle section (230). A foldable, circular spring (232) comprising, e.g., a superelastic alloy such as a member of the nitinol family, is shown. The expanded diameter of the expandable stop assembly (214), as shown in FIG. 7B, is larger than that of the delivery catheter (220).

Reference is now made to FIG. 7C which illustrates s a perspective view of the expandable stop assembly (214) having the expanded disc (226), the circular spring (232), the proximal slideable ring (228) about the core wire (222), and the sliding middle section (230).

After the embolectomy assembly (210) is properly positioned with the distal collection assembly (216) properly distal of the target clot and the proximal collection assembly (218) properly proximal of the clot, the retractable sheath (212) is retracted allowing the distal collection assembly (216), the proximal collection assembly (218), and the expandable stop assembly (214) to expand to their final diameters, the clot extraction procedure may begin. The core wire (222) is used to control the position of the distal collection assembly (216) and the delivery catheter (220) is used to control the position of the proximal collection assembly (218). The two collection assemblies (216, 218) are moved towards each other to collect or grab the clot. The delivery catheter (220) is then withdrawn from the vessel with the extracted clot.

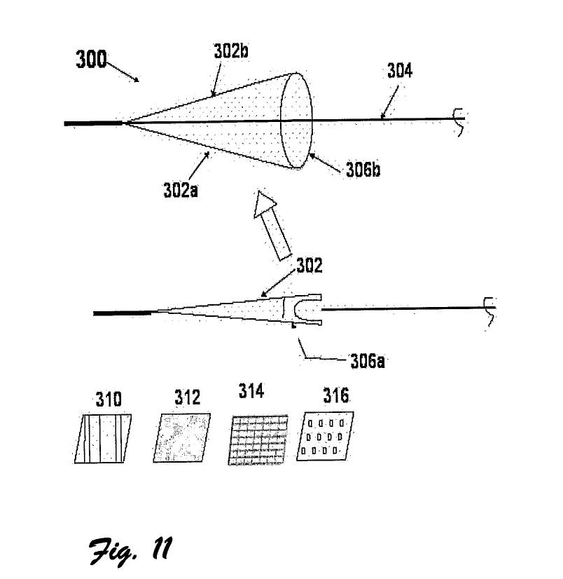

Reference is now made to FIGS. 8A-9B which provide examples of suitable structures for the distal and proximal collector assemblies.

FIG. 8A is an end view of one variation of a collector assembly (250) (i.e., the effecters) with a leading element (252) comprising a plurality of panels (253) separated by small gaps. The panels (253) may be positioned by, e.g., gluing them to one or more filaments (254) or by integrally casting or extruding the leading element (252). This element may be formed with a center section (256) associated with the panels (253) to support them and, in some variations, provide impetus for expansion. The center section (256) may be configured to slide over the core wire (e.g., 222 in FIG. 7C) or be affixed to the core wire, as appropriate in the particular design.

FIG. 8B shows a side view of the collector assembly (250) shown in FIG. 8A. The leading element (252) explained with regard to that FIG. 8A may be seen. Additionally, the trailing element (258) may be seen; the trailing element (258) comprising a filter-like material, operative to collect embolic/clot detritus passing through the leading element (252). Suitable materials for the trailing element (258) are fabrics (woven or nonwoven) polyethyleneterephthalate (PET), PTFE, ePTFE, polypropylene, or the like having sufficient flexibility to fold to the core wire (as has been discussed above) and pore size sufficient to filter soft emboli/clot from the blood. An example of a commercial material suitable for the trailing element (258) may be seen in the BALT CATCH mechanical thrombus retriever. The trailing element (258) may be attached at the outer periphery (or at other locations) suitable for collecting the thrombus particles passing through the leading element (252).

FIGS. 9A and 9B show another variation of a collector assembly (260) (also referred as the effecters), that may be either a distal or proximal collector assembly.

FIG. 9A provides a front view of a leading element (262) having openings (264) allowing passage of softer emboli/clot. FIG. 9B shows the leading element (262) and also the following element (266) for collecting emboli/clot sections passing through the openings (264) in the leading element (262). Also shown is a center section (268) having the same function as the center section discussed just above.

Again, the materials, thicknesses, and stiffnesses of the leading (262) and following element (266) are selected to allow those elements to fold to the core wire as shown above. Each of these collector assemblies may have an integral or discrete element or actuator to controllably expand the assemblies or to allow those assemblies to expand when appropriate.

As discussed in passing above, the physical structure of many emboli/clots found in the blood stream may be characterized as having several densities or physical states, ranging in description to near soft-solids to jelly-like to soft-gel-like. The device is intended to allow passage of softer materials through the openings in the leading elements of the collector assemblies for collection in the trailing elements. FIG. 10 provides a schematic representation of a leading element (272) having openings (274) allowing the softer portions (276) of a clot (278) (also having a firmer section (280)) to extrude smaller portions (282) through to the filter-like following element (284) for collection.

Reference is now made to FIG. 11 which illustrates another variation of a distal collection element (also referred as the distal effecter), to be used with a leading collection element to form a distal collection assembly.

This self-expanding distal collection assembly variation (300) includes a pair of ribs (302) extending in a generally longitudinal direction. The ribs (302) are fixedly attached to the core member (304) at the distal end and to a collapsible ring (306a in its collapsed condition and 306b in its expanded condition) at the proximal end. In another variation, one of the ribs (302) may be eliminated.

The collapsible ring (306a, 306b) desirably comprises a suitably elastic material, such as a nitinol material, having a very small diameter. Collapsible rings (306) of superelastic alloys such as nitinol may be folded proximally and distally as shown (306b) and inserted beneath a release component, such as the sheath (300) seen in FIG. 3.

The framework comprised of the ribs (302) and the proximally located collapsible ring (306) may be covered with a cone of material suitable for collecting and holding a target embolus. Biocompatible materials such as polyethylene terephthalate (PET), polytetrafluoroethylene (PTFE or Teflon), expanded PTFE (ePTFE or GoreTex), polypropylene, or the like. The form of the material may be woven fabrics (310), non-woven fabrics (312), gauze-like fabrics (316), fluid-permeable membranes, or the like. In general, the material of the cone should be selected to allow some amount of fluid passage, but retain the soft embolus.

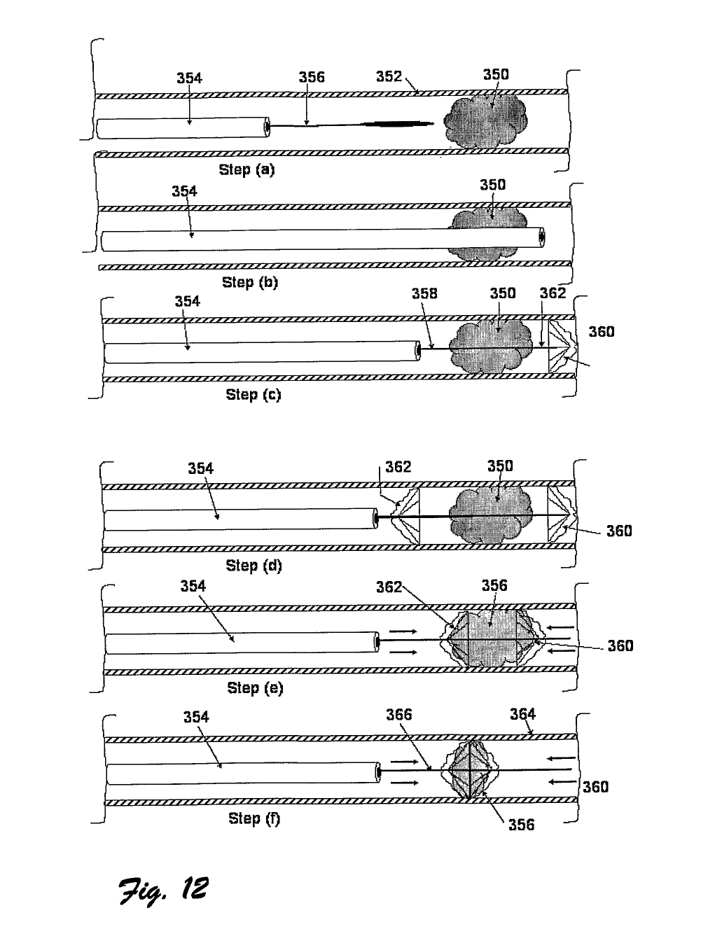

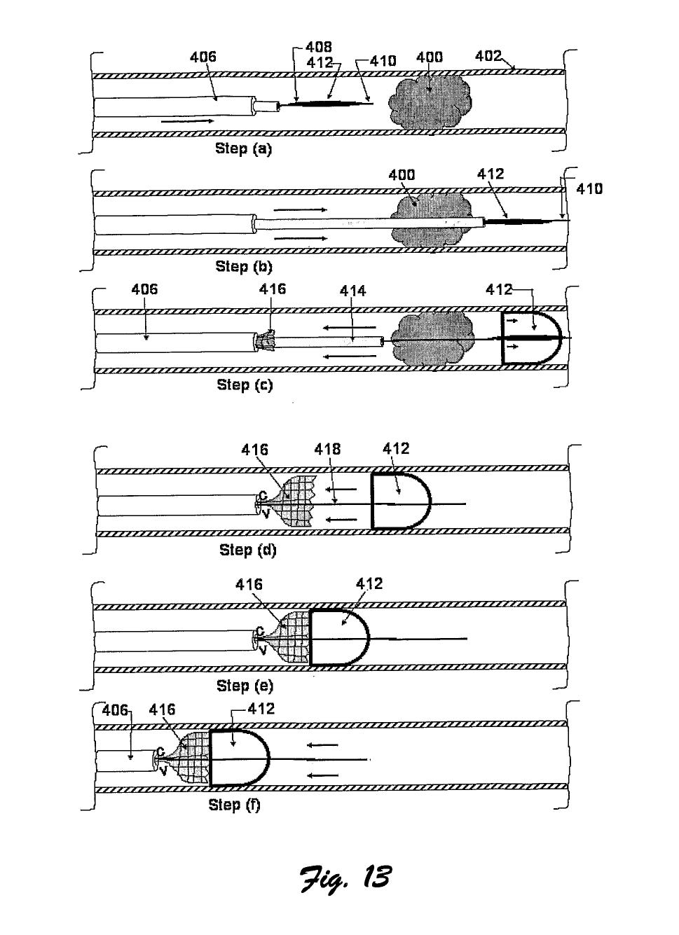

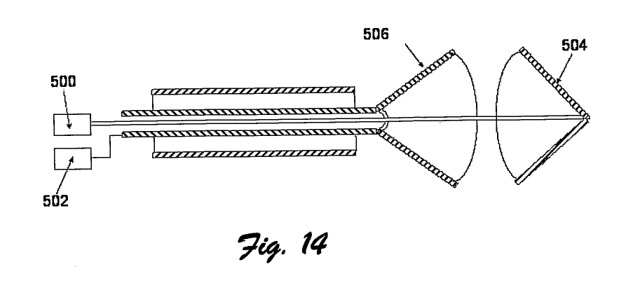

Reference is now made to FIG. 12 which schematically illustrates a typical procedure for using the variations of the embolectomy device in which the catheter penetrates the clot to remove that clot (350) from an artery (352). This procedure is also used to pass the delivery catheter between the clot and the vascular wall.

In step (a), the distal end of a microcatheter (354) has been placed in the vicinity of the target clot (350) using a guidewire (356).

In step (b), the microcatheter (354) has penetrated the clot (350) the guidewire (356) that was used to direct that microcatheter (354) has been withdrawn. After the guidewire is withdrawn, it is replaced with the embolectomy device.

In step (c) of FIG. 12, the embolectomy device (358) has been extended through the microcatheter (354) and the distal collection assembly (360) (also referred as the distal effecter), expanded. The microcatheter (354) has been withdrawn from the clot sufficiently that the catheter's distal tip is proximal of the clot (350).

In step (d), the proximal collector assembly (362) (also referred as the proximal effecter), has been extended from the distal tip of the catheter (354), has been expanded, and is progressing towards the clot (350).

In step (e), the distal collection assembly (360) and the proximal collector assembly (362) are being pulled towards each other and softer parts of the thrombus (356) are being pushed through the leading elements of the distal and proximal collector assemblies (360, 362) into the trailing elements of those assemblies.

In step (f), the clot (356) has been separated from the vascular wall (364) and collected by the embolectomy device. The catheter (354, core wire (366), and the embolectomy device (358) with enclosed thrombus (356) are all withdrawn from the blood vessel (352).

Reference is now made to FIG. 13 which illustrates s a typical procedure for using the variations of the embolectomy device in which the catheter is not used to penetrate the clot to remove that clot (400) from an artery (402).

In step (a), the distal end of a microcatheter (406) has been placed in the vicinity of the target clot (400). The guidewire used to direct the microcatheter (406) has been withdrawn and the embolectomy device (408) introduced. The embolectomy device is shown in step (a) extending from the microcatheter (406) and progressing towards the clot (400).

The nose element (410) and the collapsed distal collector assembly (412) (also referred as the distal effecter), may also be seen.

In step (b), the nose element (410) and the collapsed distal collector assembly (412) have been pushed through, and distally of, the clot (400).

In step (c), the release sheath (414) has been withdrawn allowing the distal collector assembly (412) to self expand. The proximal collector assembly (416) (also referred as the proximal effecter), is beginning to extend from the distal end of the catheter (406).

In step (d), the proximal collector assembly (416) has been extended to its full diameter. The user has begun pulling the core element (418) and distal collector assembly (412). The distal collector assembly (412) has collected the clot and is approaching the proximal collector assembly (416).

In step (e), the proximal collector assembly (416) has contacted the distal collector assembly (412) completely enveloping the embolus.

In step (f), the proximal collector assembly (416) and the distal collector assembly (412) containing the clot are withdrawn along with catheter (406). The procedure is complete.

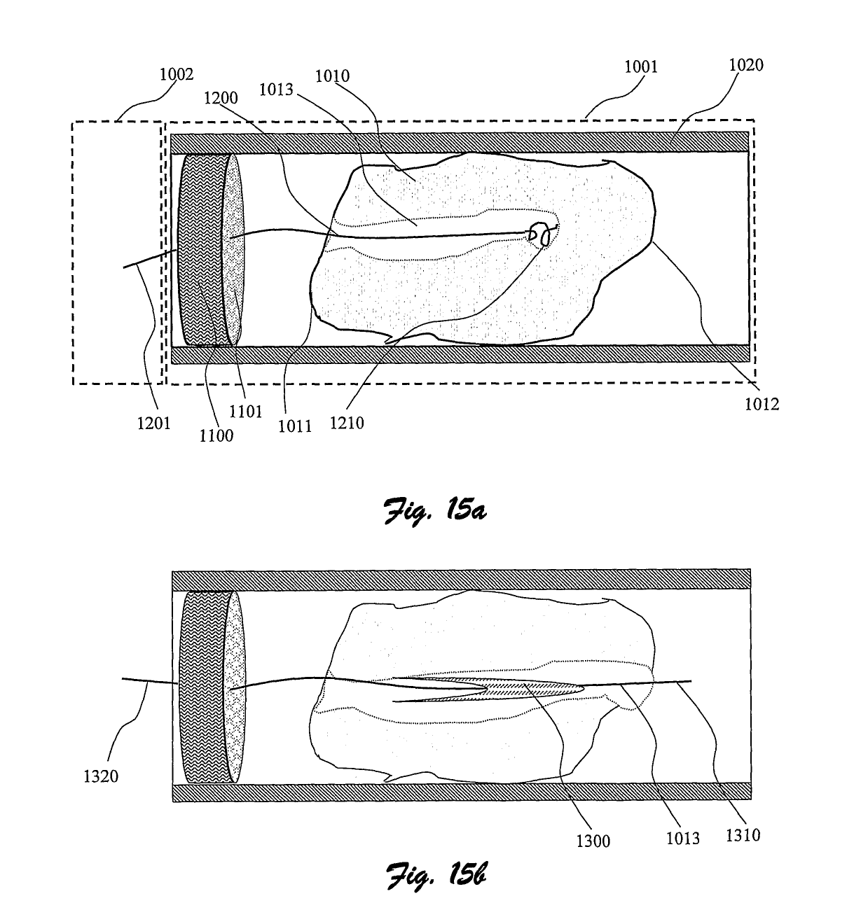

Reference is now made to FIG. 14 which illustrates an optional vibrator structure useful in removing or loosening a target clot from a vascular wall. The Figure shows, in schematic fashion, the operation of one or more vibration components (500, 502) that impose axially or radially directed vibrations ultimately onto the distal collector (504) and the proximal collector (506) (also referred as the proximal effecter),. The distal collector (504) (also referred as the distal effecter), and the proximal collector (506) may be independently oscillated or simultaneously oscillated. They may be oscillated in or out of phase. They may be operated at the same or different frequencies. Suitable frequencies are in the range of 100 Hz to about 30 kHz.

The vibration may be imposed on the distal collector (504) and the proximal collector (506) before the collectors are moved towards each other or during their passage towards each other.

In general, the vibration imposed upon the device should not be of an amplitude or frequency that tends to break up the target clot, but instead should be sufficient to improve removal of the clot from its adherence to the vascular wall.

The distal collector (504) and the proximal collector (506) are depicted as generic components for illustration of the vibration function. The vibration components (500, 502) may be used in conjunction with any of the proximal collector and distal collector designs disclosed elsewhere herein.

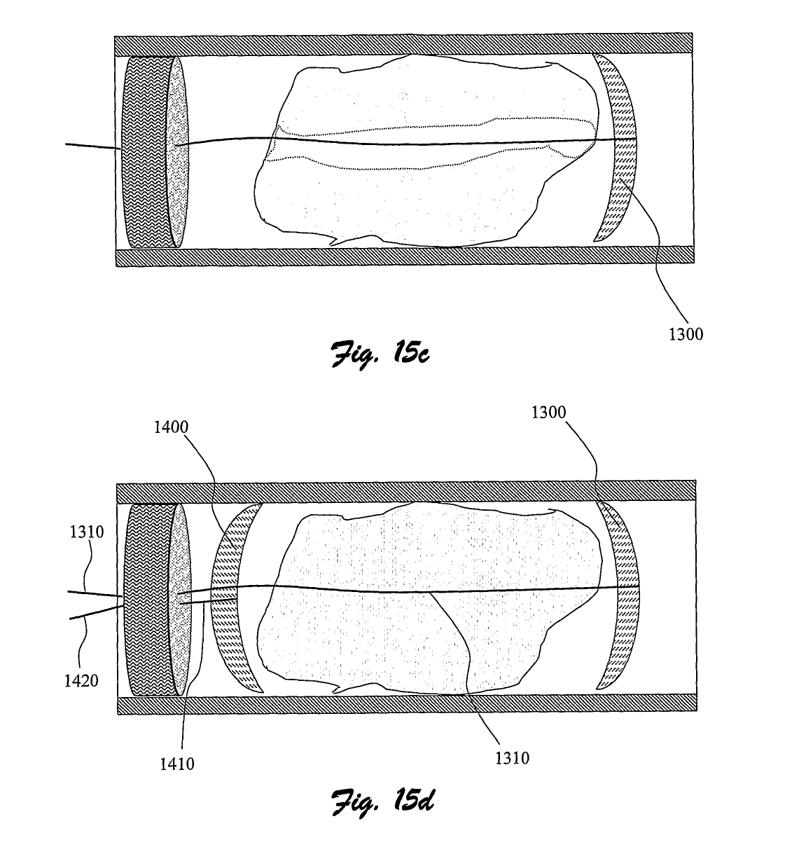

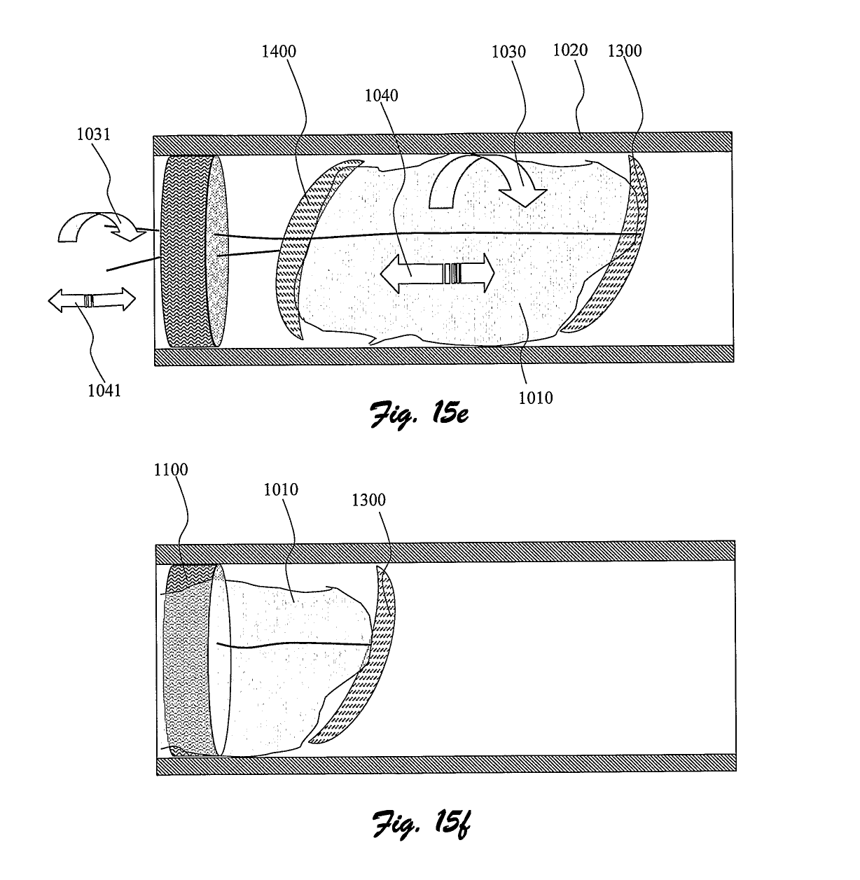

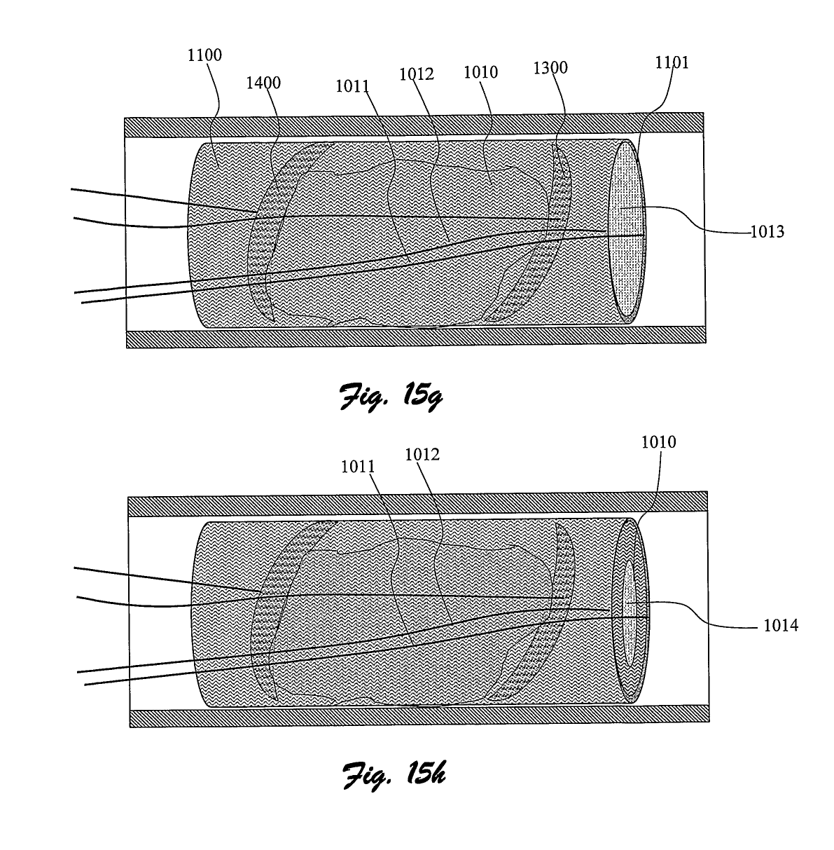

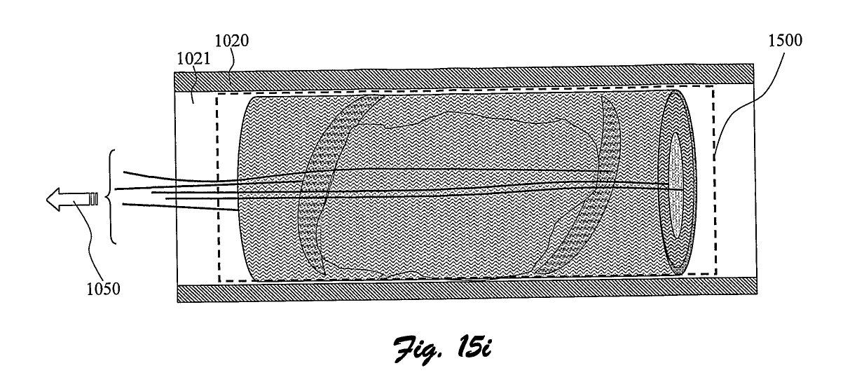

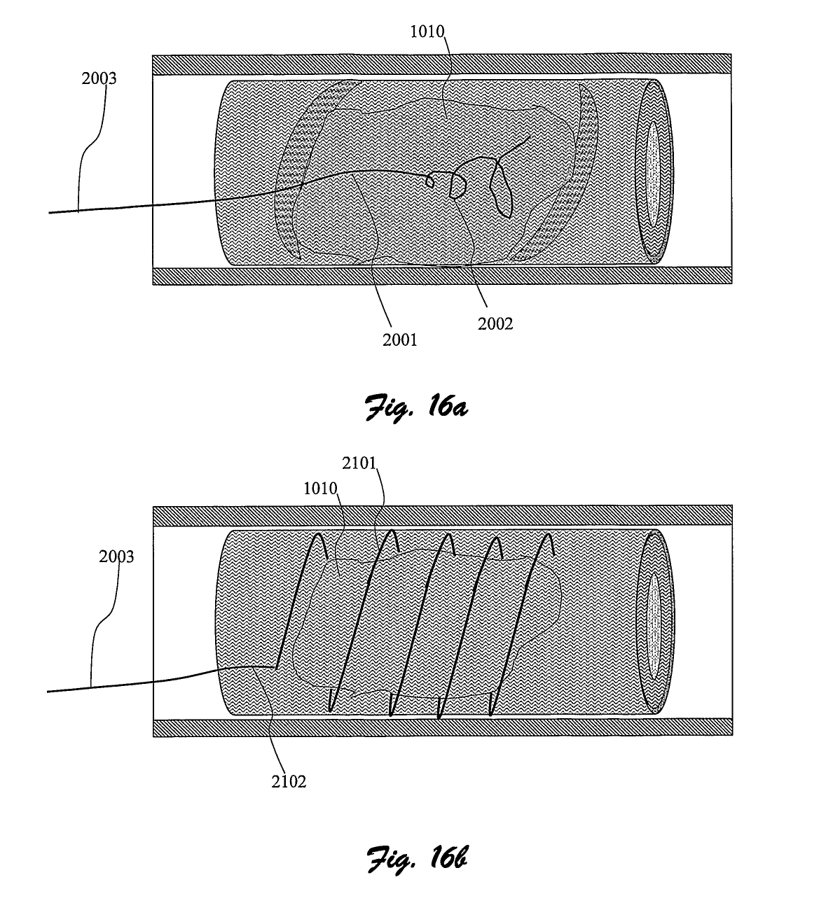

Reference is now made to FIGS. 15a to FIG. 15i, presenting another view in an out-of-scale manner a schematic view (cross section) the minimally invasive embolectomy device according to another embodiment of the invention.

As described above, the device is designed to extract clot (1010) found in the human vasculature (1020), particularly in the distal, tortuous neurovasculature (location 1001, within the body of the patient), with a low or minimized pulling or extractive force from location 1002, outside the body of the patient. The device is adapted to (i) reversibly grasp clot (1010) between two opposing, multi-element collection assemblies, i.e., distal clasp (1300) and proximal clasp (1400) and, in doing so, separates the clot from the vascular wall; (ii) to insert clot (1010) within of net (1100) via its the distal inlet (1101); and then, (iii), when clot (1010) is secured within net (1100), extracting enveloped clot form within the body (location 1001) outside the body (location 1002).

FIG. 15a illustrates clot (1010) in an occluded vascular lumen (1020). A net-like graft (1100) is inserted within lumen (1020), e.g., in a percutaneous manner, such that its distal end is located adjacent the embolus. Guidewire 1200, operated from its proximal portion 1201, is inserted via graft 1100 and manipulated to cross the embolus, e.g., by means of an effecter 1210 located in its very distal end.

The effecter is selected, in a non-limiting manner, from a group consisting of a coil-like, screw-like or drill-like head, reciprocally and/or rotatably operated in one or by a sequence of maneuvers.

According to one possible embodiment of the invention, a small-diameter bore 1013 is provided within clot 1010.

According to yet another embodiment, guidewire 1200 crosses the clot without forming a noticeable bore.

FIG. 15b illustrates yet another embodiment of the invention, wherein distal clasp (1300) is reversibly folded to a thin arrow-like member, crossing the clot by means of a guidewire. According to this embodiment, distal clasp (i.e., distal effecter) (1300) has a non unexpanded -configuration, e.g., an arrow-like spiral wound or otherwise collapsed member of a respectively thin cross section and narrow circumference; and an expanded -configuration, e.g., a canopy, cymbal or net-like member of a wider cross section and longer circumference.

The endless guidewire 1013 is operated in its proximal portion (1320) outside the body of patient, and exceeds to the embolus's distal end (1310).

FIG. 15c illustrates distal clasp (1300) in its expanded configuration. The diameter of the clasp, in this illustration, is adapted to fit in size and shape the inner diameter of the blood vessel 1020.

FIG. 15d illustrates both distal clasp (1300) and proximal clasp (1400) (i.e., proxima; effecter) in their expanded configuration. Distal clasp and proximal clasp are manipulated by means of at least one operating wires 1310 and 1420, respectively.

FIG. 15e illustrates a possible mode of action of the device especially adapted to (i) reversibly grasp clot (1010) between the two opposing distal clasp (1300) and proximal clasp (1400). One or both of the clasps are manipulated, e.g., either linearly (1041) or rotatably (1031) by means of operating wires in, e.g., (i) a single reciprocal stroke along the long longitudinal axis of the blood vessel, wherein clot is maneuvered in a reciprocate facilitated motion 1040; (ii) a set of reciprocal motions, for example in-and-out opposite motions, a vibrating set of reciprocal motions, etc.; (iii) a single rotating stroke around the long longitudinal axis of the blood vessel, wherein clot is maneuvered in a circle facilitated motion 1030; (iv) a set of rotational motions, for example circular opposite motions, a vibrating set of circular motions, etc.; (v) vibration motions; and, (vi) any combination of the same. By those maneuvers, the clot is separated from the vascular wall 1020.

FIG. 15f illustrates the step wherein clot (1010) which was separated from the vascular wall is inserted within net (1100) via its distal inlet (1101), by pulling distal clasp (1300) inside the net.

FIGS. 15g and 15h illustrate each a secured physical conjugate of an enveloping net 1100, containing distal and proximal clasps (1300 and 1400, respectively) that immobilize clot 1010 within the structure. According an embodiment of the invention, distal inlet of the net (1101) is a constricting sphincter-like orifice. The diameter of orifice is regulated, e.g., by means of operating wires 1011 and 1012, by electrical charge (in case of Nitinol made sphincters) by electro-active polymers etc. By pulling wires 1011 and 1012, or by activating the distal portion of the net 1010, the diameter of the orifice is reduced, e.g., from the expanded configuration 1013 to semi close configuration 1014, or to a totally close configuration (nt shown here).

FIG. 15i illustrates a last step, in which enveloped clot (see conjugate 1500) is safely extracted form within the body (location 1001, FIG. 15a) outside the body (location 1002). According to one embodiment of the invention, all operating wires are pulled (1050) and clasped clot 1500, now free from its native vascular wall 1020 is pulled via predetermined track 1021 outside of the patent body.

It is according to yet another embodiment of the invention wherein an inner-coil assembly is utilized. Reference is now made to FIG. 16a, presenting a corkscrew-like effecter, having a coil-like distal end, adapted to effectively anchor the clot from its inner portion, and an endless wire 2001 which is terminated in a operating portion of the effecter, locate in the very proximal end, namely outside the body of the patient. It is in the scope of the invention wherein the corkscrew-like effecter is utilized either with or without one or more of the distal and proximal clasps. It is according to another embodiment of the invention wherein an outer-coil assembly is utilized. Reference is now made to FIG. 16b, presenting a spool, having a coil-like distal end, adapted to effectively anchor the clot from its outer surface, and an endless wire 2101 which is terminated in a operating portion of the effecter 2003, locate in the very proximal end.

It is further in the scope of the invention wherein the spool is utilized either with or without one or more of the distal and proximal clasps.

It is acknowledged in this respect that distal and proximal clasps as defined in any of the above are provided useful to effectively clasp a clot from its both distal and proximal surfaces in an opposite cymbal-stroking coordinated manner and thus to (i) free the clot from its surrounding vascular wall by applying reciprocal/rotational maneuvers; (ii) enforce the clot towards the net's distal inlet in a safe manner, i.e., in a way the clot does not disengage to small pieces and does not escape, as a whole, downstream away from the net; and (iii), positioned the embolus, as is, within the net and providing a proximal-distal structural embolus-grasping element in a safe manner, i.e., in a way the clot does not disengage to small pieces and does not escape, as a whole, downstream away from the net.

Also is acknowledged that each and both the corkscrew-like effecter implanted within the embolus, and the spool warping the outer circumference of the same, are adapted to apply a preset and well directed force, and to grasp the clot in a vector which is approximately parallel to its main longitudinal axis, namely a force applied from and towards the embolus's distal and/or proximal poles. Hence, both internal and external coiled effecters are provided useful to (i) free the clot from its surrounding vascular wall by applying reciprocal/rotational maneuvers; (ii) enforce the clot towards the net's distal inlet in a safe manner, i.e., in a way the clot does not disengage to small pieces and does not escape, as a whole, downstream away from the net; and (iii), positioned the embolus, as is, within the net and providing a proximal-distal structural embolus-grasping element in a safe manner, i.e., in a way the clot does not disengage to small pieces and does not escape, as a whole, downstream away from the net.

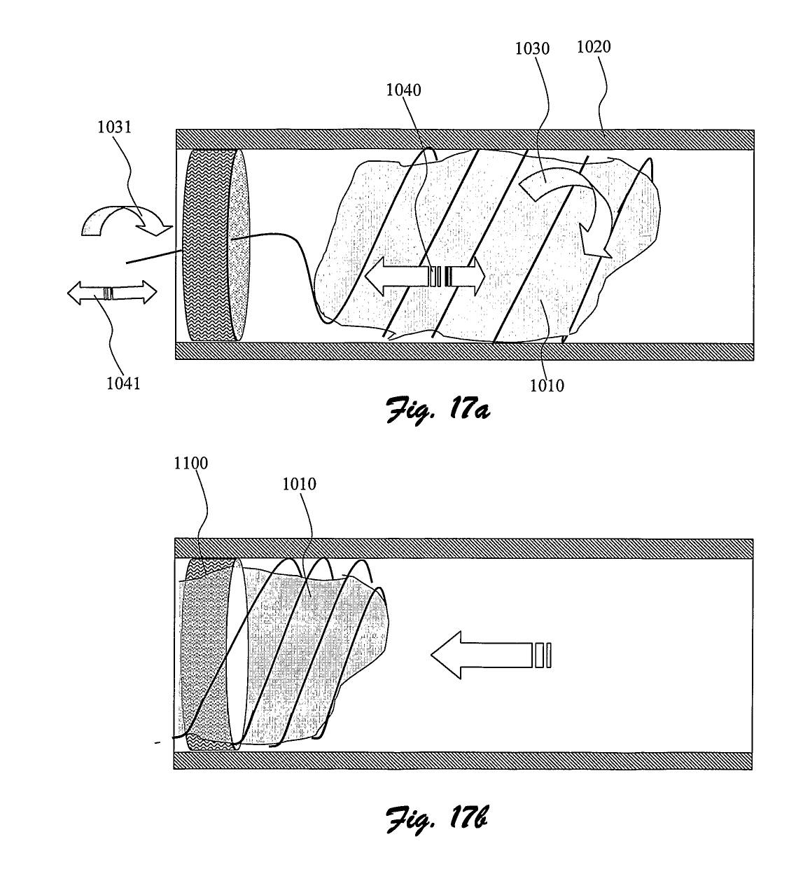

Reference is thus made to FIGS. 17a and 17b which illustrates an externally embolus-warping coil-like effecter, characterized by a distal spool portion in a physical connection with a proximal operating wire. By applying, by means of the proximal end, linear reciprocating movement and/or rotational maneuvers (1041 and 1031, respectively), clot 1010 reciprocate and/or circularly actuate (1040 and 1030, respectively), until it separated from vascular wall 1020. Consequently, as illustrated in FIG. 17b, working wire is pulling the spool, which in return pulls the clot (1010) into the net (1100) in distal to proximal linear motion.

It should be emphasized that the embolus-warping coil-like effecter is provided in combination with the proximal effecter and the distal effecter.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1-44. (canceled)

45. An embolectomy device for removal of clots from vasculature, said device comprising:
a. a proximal effecter characterized by a non-expanded configuration and an expanded configuration;
b. a distal effecter characterized by a non-expanded configuration and an expanded configuration;
said proximal effecter, in said expanded configuration is adapted for grasping a proximal portion of the target embolus; said proximal effecter, in said expanded configuration is adapted for grasping a distal portion of said target embolus;
wherein both said effecters, when expanded, are oppositely positioned and are adapted to operate in concert, for trapping said clot such that said clot is (i) manipulatable along and/or around the main longitudinal axis of said vasculature in a predetermined set of motions; (ii) extracted out of said vasculature.

46. The embolectomy device of claim 45, wherein said distal effecter is operative to cross a target clot in said non-expanded configuration and then to be expanded to said expanded configuration.

47. The embolectomy device of claim 45, wherein said distal effecter is operative to self-expand from its non-expanded configuration to its expanded configuration.

48. The embolectomy device of claim 45, further comprising a covering net, adapted to accommodate said grasped clot and to extract the same outside the body of the patient.

49. The embolectomy device of claim 49, wherein said net is selected from the group consisting of woven fabrics, non-woven fabrics, gauze-like fabrics, materials having multiple openings, and fluid-permeable membranes.

50. The embolectomy device of claim 49, wherein the distal portion said net is adapted to switch between an expended-configuration to a restricted-configuration; said expended-configuration is operative to allow insertion of at least a portion of a target clot into said net's distal portion; and said restricted configuration is operative to retain said clot within said net.

51. The embolectomy device of claim 49, wherein said distal portion of said net is operative to self-expand from its expended-configuration to its restricted-configuration.

52. The embolectomy device of claim 49, wherein said net is an elongated sleeve comprising a restricting sphincter located at the distal end of said sleeve, adapted to retain at least a portion of the target clot within said net.

53. The embolectomy device of claim 45, wherein said distal effecter is incorporated or otherwise connected with said proximal effecter to form a single effecter.

54. The embolectomy device of claim 45, wherein said set of motion is selected from a group consisting of (i) a single reciprocal stroke along the long longitudinal axis of said blood vessel; (ii) a set of reciprocal motions; (iii) a single rotating stroke around said long longitudinal axis of said blood vessel; (iv) a set of rotational motions; (v) vibrating motions; or any combination of the same.

55. The embolectomy device of claim 45, additionally comprising:
an endless wire coupled to a spring-like helical member having a plurality of loops; said loops are positioned at an angle A relatively to the main longitudinal axis of said blood vessel; and, are adapted to radially encircle at least a portion of the outer circumference of said clot; and,
a tubular mesh-like net for both enveloping said helical member and enclosing said clot therein;
wherein at least a portion of said clot is contemporaneously confined within said loops and said mesh-like net, such that radial compression forces and longitudinal shearing forces are exerted on said clot and the tendency of said clot to fragment is mitigated.

56. The embolectomy device of claim 55, wherein said spring-like helical member and/or said mesh-like net comprising means for applying vibration on said clot.

57. The embolectomy device of claim 55, wherein said mesh-like net is made of material selected from a group consisting of woven fabrics, non-woven fabrics, gauze-like fabrics, materials having multiple openings, and fluid-permeable membranes or any combination thereof.

58. The embolectomy device of claim 55, wherein said mesh-like net additionally comprising materials selected from a group consisting of woven fabrics, non-woven fabrics, gauze-like fabrics, materials having multiple openings, and fluid-permeable membranes or any combination thereof.

59. The embolectomy device of claim 55, wherein said spring-like helical member is coupled to said mesh-like net.

60. The embolectomy device of claim 55, wherein said distal portion of said mesh-like net is adapted to switch from an expended-configuration to a restricted-configuration; said expended-configuration is operative to allow insertion of at least a portion of a target clot in to said mesh-like net's distal portion; and said restricted configuration is operative to retain said clot within said mesh-like net.

61. The embolectomy device of claim 55, wherein said distal portion of said mesh-like net is operative to self-expand from its expended-configuration to its restricted-configuration.

62. The embolectomy device of claim 55, wherein said mesh-like net is an elongated sleeve comprising a restricting sphincter located at the distal end of said sleeve, adapted to retain at least a portion of the target clot within said mesh-like net.

63. The embolectomy device of claim 55, wherein said helical member is characterized by a cross-sectional area selected from a group consisting of circular, oval, round, square, rectangular, triangular, regular or irregular shapes, or any combination thereof.

64. The embolectomy device of claim 55, additionally comprising a motor adapted to rotate said helical member.

65. The embolectomy device of claim 55, wherein said helical member additionally comprising protrusions for assisting in the separation of said clot from said vasculature.

66. The embolectomy device of claim 45, additionally comprising vibration means applied on said clot.

67. The embolectomy device of claim 45, additionally comprising a corkscrew-like effecter, having a coil-like distal end, adapted to penetrate and cross said clot.

68. The embolectomy device of claim 55, wherein the diameter of said helical member is variable so as to better adjust to the diameter of said clot or said blood vessel.

69. The embolectomy device of claim 55, wherein said angle A ranges from about 0 degrees to about 180 degrees.

70. The embolectomy device of claim 45, additionally comprising aspiration means.

71. The embolectomy device of claim 55, wherein at least one of said loops and/or said mesh-like net is coated with lubricious polymeric material selected from hydrophilic polymer material.

72. The embolectomy device of claim 55, wherein said spring-like helical member is characterized by single, double, or triple pitch.

73. The embolectomy device of claim 55, wherein the cross section of at least one of said loops and/or said mesh-like net varies.

74. A method for removing a clot from blood vessel, comprising steps of:
i. obtaining an embolectomy device comprising:
a. a proximal effecter characterized by a non-expanded configuration and an expanded configuration;
b. a distal effecter characterized by a non-expanded configuration and an expanded configuration;
ii. approximating said clot;
iii. crossing said clot via said distal effecter in said an expanded configuration;
iv. reconfiguring said distal effecter from said an expanded configuration to said expanded configuration;
v. reconfiguring said proximal effecter from said an expanded configuration to said expanded configuration;
vi. grasping both the proximal portion of said clot via said proximal effecter and the distal portion of said clot via said distal effecter;
vii. manipulating said clot along and/or around the main longitudinal axis of said blood vessel in a predetermined set of motions; thereby separating said clot from the walls of said blood vessel; and,
viii. extracting said clot confined from said blood vessel.

75. The method according to claim 74, additionally comprising step of selecting said set of motion from a group consisting of a (i) single reciprocal stroke along the long longitudinal axis of said blood vessel; (ii) a set of reciprocal motions; (iii) a single rotating stroke around said long longitudinal axis of said blood vessel; (iv) a set of rotational motions; (v) a vibration motions; or any combination of the same.

76. The method according to claim 74, additionally comprising steps of crossing said clot via said distal effecter in said en-expanded configuration; and, expanding to said expanded configuration.

77. The method of claim 74, additionally comprising step of configuring said distal effecter to self-expand from its un expanded configuration to its expanded configuration.

78. The method of claim 74, additionally comprising step of incorporating said distal effecter with said proximal effecter to form a single effecter.

79. The method according to claim 74, additionally comprising step of selected the cross sectional area of said helical member from a group consisting of circular, oval, round, square, rectangular, triangular, regular or irregular shapes, or any combination thereof.

80. The method according to claim 74, additionally comprising step of penetrating and crossing said clot via a corkscrew-like effecter.

81. The method according to claim 74, additionally comprising step of accommodating said grasped clot via a covering mesh-like net.

82. The method according to claim 81, additionally comprising step of selecting the material of said mesh-like net from a group consisting of woven fabrics, non-woven fabrics, gauze-like fabrics, materials having multiple openings, and fluid-permeable membranes or any combination thereof.

83. The method according to claim 81, additionally comprising step of configuring the distal portion of said mesh-like net to switch from an expended-configuration to a restricted-configuration; said expended-configuration is operative to allow insertion of at least a portion of a target clot into said mesh-like net's distal portion; and said restricted configuration is operative to retain said clot within said mesh-like net.

84. The method according to claim 81, additionally comprising step of configuring said mesh-like net to self-expand from said expended-configuration to said restricted-configuration.

85. The method according to claim 81, additionally comprising step of retaining at least a portion of the target clot within said mesh-like net.

86. The method according to claim 74, additionally comprising step of applying vibration on said clot.

87. The method according to claim 74, additionally comprising step of adjusting the diameter of either one of said effecters to the diameter of said clot.

88. The method according to claim 81, additionally comprising step of coating at least one selected from a group consisting of said mesh-like net and/or said proximal effecter and/or said distal effecter with lubricious polymeric material selected from hydrophilic polymer material.
